(19) United States
(12) Patent Application Publication
(10) Pub. No.: US 2026/0137473 A1
(43) Pub. Date: May 21, 2026

(54) MULTI-LAYER C-ARM DRAPE

(71) Applicant: Medline Industries, LP, Northfield, IL (US)

(72) Inventors: Kasey Laine COCKERILL, Atlanta, GA (US); Jason Owen HACKETT, Canton, GA (US); Rebecca Elise FISHER, Moreland, GA (US); Steffanie T. MATTHEWS, Cumming, GA (US); Tracy Joseph WHITE, Cumming, GA (US); Edwin Michael GEORGE, Duluth, GA (US)

(21) Appl. No.: 18/951,441

(22) Filed: Nov. 18, 2024

(51) Int. Cl. A61B 46/10; A61B 46/00

(52) U.S. Cl. CPC A61B 46/10; A61B 46/40

(57) ABSTRACT

A drape, such as but not limited to, a drape that can be used to cover a C-arm of a medical device is provided. The drape has multiple layers, including a base layer and one or more removable layers. A tether is associated with each removable layer for separately removing each removable layer. The removable layers allow for repeated introduction of an imaging unit into the sterile zone of a surgical theater.

### FIELD OF THE INVENTION

The present invention relates generally to surgical drapes for use with medical imaging using a C-arm, and particularly to a surgical drape having multiple removable layers.

### BACKGROUND

C-arms are commonly used during surgery to provide real-time static or dynamic fluoroscopic imaging. Medical equipment utilized in patient care facilities is susceptible to contamination from bodily fluids. It can be important to effectively remove these bodily fluids from the equipment surfaces in order to maintain a relatively sterile environment within the facilities. This provides advantages for preventing the exposure of both patients and healthcare personnel to potential communicable diseases that can be transmitted through bodily fluids. While drapes are commonly used to cover the equipment, their application can result in unintended contamination.

The importance of maintaining a sterile environment in a surgical theater is directly linked to the known risk of infections caused by bacteria, whether originating from individuals or surgical equipment within the facility. Consequently, minimizing the risk of infection during surgical procedures requires preventing the transfer of bacteria through airborne lint or dust particles, fluids, or other means within the surgical theater. Despite the well-documented risks associated with surgical infections, preventing their occurrence remains a significant challenge.

One strategy employed to inhibit surgical site infections involves using drapes to protect against organisms that may be present. However, current draping techniques may involve the use of a “half sheet,” which is a plain rectangular sheet of material draped over equipment in the operating theater. While this type of drape may be effective for operations not involving relatively large, specialized surgical equipment, it may not be suitable in cases where unsterilized surgical equipment, such as radiological imaging equipment like a C-arm fluoroscopy unit, needs to be introduced into the operating room for use during surgical procedures.

In light of the large, arcuate design of the C-arm fluoroscopy unit and its mobility during usage, maintaining sterility in the surgical theater poses a significant challenge. Typically, multiple half sheets are utilized, albeit in a cumbersome manner, to cover the unit during procedures. This may lead to procedural delays or necessitate less effective approaches as surgical teams attempt to position the sheets around different parts of the unit, especially the arm and imaging end. The absence of a universally accepted draping methodology for C-arm radiological units further accentuates the need for creativity. Inadequate deployment of the half sheets compromises the sterility of the surgical theater, consequently elevating the risk of patient infection. Furthermore, due to the repeated movement of this radiological equipment during surgery, there is an inherent risk that the current draping mechanisms may shift, exposing unsterilized areas of the unit. In particular, because of the manner of movement of the C-arm, portions of the arm may be required to repeatedly enter, exit, and reenter the sterile field.

Therefore, there is a pressing need for enhanced sterile drapes and a method to establish and sustain sterility in a surgical theater, particularly for the use with relatively large radiological imaging equipment such as C-arm radiological units, including lateral positioning of C-arm units.

### SUMMARY OF ILLUSTRATIVE EMBODIMENTS

Embodiments of a drape for use with a C-arm imaging unit may comprise a base layer covering a surface of an imaging head of the imaging unit. The imaging head may be moveable between a retracted position outside a sterile zone and an extended position inside the sterile zone. The drape may further comprise first removable layer at least partially covering the base layer, and the first removable layer may comprise a first tether extending from the first removable layer. The first removable layer may be removed from the base layer by pulling on the first tether in conjunction with movement of the C-arm imaging head from the retracted position to the extended position. In further embodiments of a drape, removing the first removable layer from the base layer may expose a sterile surface of the base layer in conjunction with the imaging head moving into the sterile zone.

Embodiments of a drape are also contemplated where the base layer is secured to the C-arm and where the drape comprises a tether attachment with a first end connected with the first removable drape and a second end detachably connected to a tether attachment. The tether attachment may be connected to the C-arm at a position spaced apart from the imaging head, which may be outside the sterile zone.

Embodiments of the drape may also comprise at least a second removable layer at least partially covering the first removable layer, and the second removable layer may comprise a second tether. The second removable layer may be removed from the base layer by pulling on the second tether in conjunction with movement of the C-arm imaging head from the retracted position to the extended position in a first movement of the imaging head into the sterile zone. Subsequently, the first removable layer may be removed from the base layer by pulling on the first tether in conjunction with movement of the C-arm imaging head from the retracted position to the extended position in a second movement of the imaging head into the sterile zone.

Embodiments of the drape may have a base layer that comprises a substantially flat sheet of flexible material and a first removable layer that comprises a substantially flat sheet of flexible material. Alternatively, the base layer may comprise a flexible material having a first seam to at least partially conform the base layer to a shape of the imaging head, and the first removable layer may comprise a flexible material having a first seam to at least partially conform the base layer to a shape of the imaging head. In embodiments of the drape, the first removable layer may be attached to the base layer by frangible connection, and the frangible connection may comprise a perforation formed in the first removable layer. In addition, the first tether may be attached to the first removable layer, and/or the first tether may be integrally formed with the first removable layer.

Embodiments of a drape for use with a C-arm imaging unit may comprise a base layer covering a surface of an imaging head of the imaging unit, where the imaging head is moveable between a retracted position outside a sterile zone and an extended position inside the sterile zone. The drape may further comprise a first or inner removable layer at least partially covering the base layer, the first removable layer comprising a first tether extending from the first removable layer, and a second or outer removable layer at least partially covering the first removable layer, the second layer comprising a second tether. In embodiments of the drape, the second removable layer may be removed from the base layer by pulling on the second tether in conjunction with movement of the C-arm imaging head from the retracted position to the extended position in a first movement of the imaging head into the sterile zone. The imaging head may then be moved to the retracted position outside the sterile zone, and the first removable layer may be removed from the base layer by pulling on the first tether in conjunction with movement of the C-arm imaging head from the retracted position to the extended position in a second, subsequent movement of the imaging head into the sterile zone, thus revealing an underlying sterile layer.

### BRIEF DESCRIPTION OF THE DRAWINGS

The foregoing and other advantages of the invention will become apparent upon reading the following detailed description and upon reference to the drawings.

FIG. 1 shows a perspective an embodiment of a surgical C-arm radiological imaging unit as used in a surgical theater.

FIG. 2 shows a side view of a surgical C-arm radiological imaging unit as used in a surgical theater.

FIG. 3A shows a side view of an embodiment of a surgical C-arm radiological imaging unit in a first, vertical position.

FIG. 3B shows a side view of the C-arm unit of FIG. 3A in a second, horizontal position.

FIG. 4A shows a cross-sectional view of an embodiment of a multi-layer drape for a C-arm imaging unit with a second or outer removable layer, a first or inner removable layer, and a base layers.

FIG. 4B shows the multi-layer drape of FIG. 4A with the second or outer layer removed.

FIG. 4C shows the multi-layer drape of FIG. 4A with the second or outer and first or inner layers removed.

FIG. 5A shows a rear view of an embodiment of a multi-layer drape with tethers positioned in a first configuration.

FIG. 5B shows the multi-layer drape of FIG. 5A with tethers positioned in a second configuration.

FIG. 6A shows a side view of an embodiment of a surgical C-arm radiological imaging unit in a first, vertical position.

FIG. 6B shows a side view of the C-arm unit of FIG. 6A in a transition position.

FIG. 6C shows a side view of the C-arm unit of FIG. 6A in a final, horizontal position.

FIG. 7A shows a perspective view of an embodiment of a tether attachment point.

FIG. 7B shows a perspective view of an alternate embodiment of a tether attachment point.

FIG. 8 shows a perspective view of a multi-layer drape positioned over an imaging head.

FIG. 9 shows a plan view of a multi-layer drape in a flat, unfolded configuration.

FIG. 10 shows perspective view of the multi-layer drape of FIG. 9 as positioned over an imaging head.

The drawings listed above are intended to convey to one of ordinary skill in the art the present invention and its embodiments. In some drawings certain elements have not been shown for clarity. While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. The following description of various embodiments, as represented in the figures, is not intended to limit the scope of the present disclosure but is merely representative of various embodiments. Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Referring to the drawings, like numbers generally indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, proximal and distal, posterior and anterior, inner and outer, and the like may be used solely to distinguish one entity or action from another entity or action without necessarily requiring or implying any actual such relationship or order between such entities or actions.

Additionally, instances in this specification where one element is “coupled to another element” can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Coupling, and securing may comprise various mechanism, including, but not limited to, adhesive, tape, bonding, or integral formation. Additionally, as used herein, “adjacent” does not necessarily denote contact. For example, one element can be adjacent to another element without being in contact with that element. In addition, the term “clinician” is used herein to indicate the medical practitioner or any other person that may utilize equipment or conduct procedures as described herein. The term includes doctors, nurses, attendants, or other medical personnel and is not intended to limit the scope of the described or claimed embodiments.

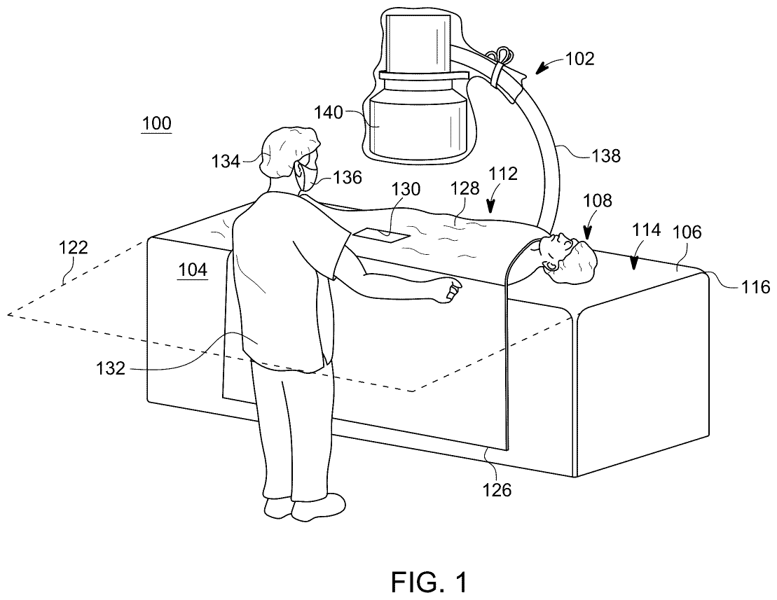

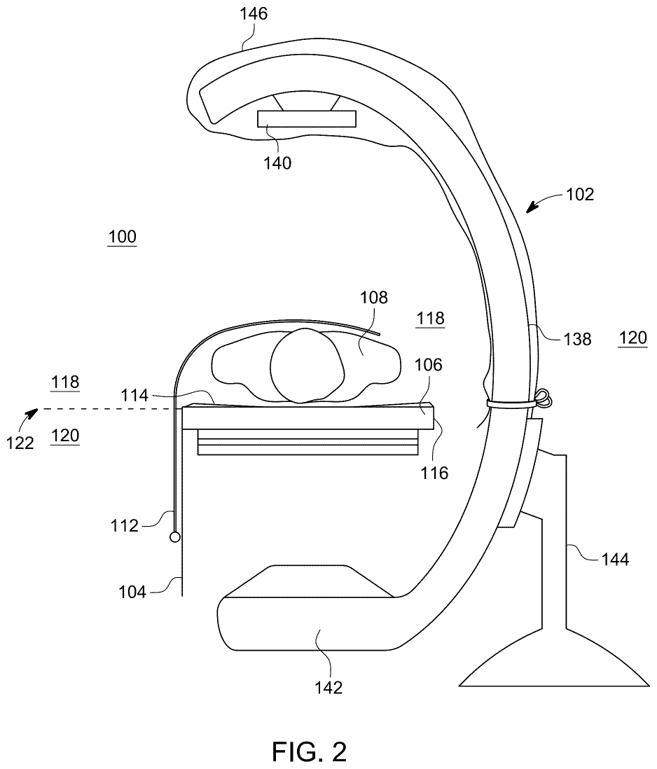

FIGS. 1 and 2 illustrate an embodiment of a surgical C-arm radiological imaging unit 102 as used in a surgical theater 100. The surgical theater may employ a drape 104 covering a table 106 in which a patient 108 is placed. In addition, a patient drape 112 may cover the patient and hang downwardly below an upper surface 114 of the table 106.

The upper surface 114 of the table 106 may delineate a sterile zone 118 above the upper surface 114 and a non-sterile zone 120 below the upper surface 114 as illustrated by line 122. The sterile zone 118, in order to reduce risk of infection to the patient 108, must remain sterile throughout the surgical procedure, and thus, extends to all equipment positioned on or above the upper surface 114 of the surgical table 106. Accordingly, any instruments within the sterile zone 118 must either be thoroughly sterilized, such as is commonly performed with smaller surgical instruments in an autoclave, or otherwise covered with a sterile barrier. In addition, any surgeon 132 or other personnel entering the sterile zone must follow sterile procedures and use appropriate clothing 134 and masking 136 to maintain the sterile environment. In contrast personnel remaining in the non-sterile zone 120 may have lesser sterility requirements.

The patient drape 112 may comprise a generally rectangular peripheral shape, with free edges 126 hanging below the upper surface 114 into the non-sterile zone 120. A central area 128 of the drape 112 is configured to remain above the upper surface 114, draped over the patient 108. The drape 112 may be provided with an opening 130 located in a predetermined position, wherein the opening 130 provides direct access to an incision surgical site on the patient 108.

The C-arm type radiological unit 102, also referred to as a C-arm fluoroscopy unit, and referred to hereafter the C-arm, may be used in surgical procedures that require multiple images to be taken of the patient from a variety of angles throughout the surgical procedure, such as a during spinal and orthopedic surgery, for example. The C-arm, as the name implies, has generally C-shaped, arcuate arm 138 that extends between a proximal, also referred to as upper, imaging head 140 and a distal, also referred to as lower, imaging head 142. The upper imaging head 140 may be an image intensifier, and the lower imaging head 142 may be an X-ray tank. The upper imaging head 140 may be covered with a sterile drape 146.

The arcuate arm 138 has a sufficient length such that during the surgical procedure, the C-arm 102 can extend beneath the operating table 106 from one side of the table 106 to an opposite side of the table 106, wherein the C-arm 102 can be readily moved, as desired, to obtain the necessary images of the patient 108. The arcuate arm 138 may be supported by a base 144 that supports the arcuate arm 138 and allows it to rotate along the arc. The C-arm unit may further comprise a control unit and one or more monitors that show the imaging results (not shown). The lower imaging head 142 may comprise a transmitter or X-ray source. The upper imaging head may comprise a receiver that detects the X-rays after they have passed through the patient so that the C-arm unit can convert the X-rays into images that can be displayed on the monitor.

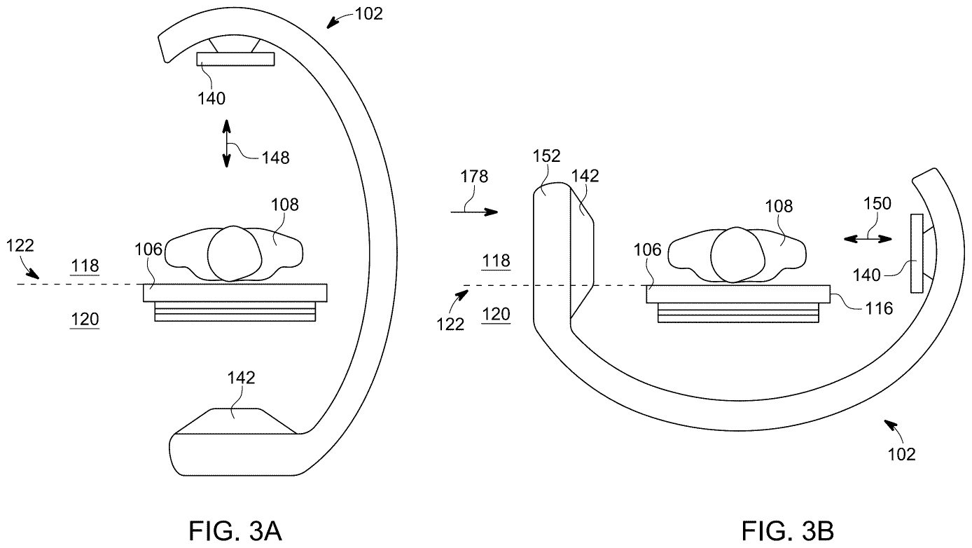

As illustrated in FIGS. 3A and 3B, it may be advantageous to rotate the C-arm 102 during a surgical procedure in order to allow imaging from a first direction, as shown in FIG. 3A to a different direction, such as a lateral direction as shown in FIG. 3B. In the initial position, FIG. 3A, the upper imaging head 140 is above the patient 108 opposite the lower imaging head 142 positioned below the patient such that the view is vertically through the patient as illustrated by arrow 148. The C-arm 102 may be rotated to a subsequent position, FIG. 3B, such that the upper imaging head is directed toward the rear portion 116, away from the surgeon, of the table 106 and side of the patient 108 as shown by arrow 150. However, when the C-arm 102 is rotated in this manner, at least a portion 152 of the lower imaging head 142 moves above the separation line 122 into the sterile field 118. Accordingly, the lower imaging head will need to be appropriately draped to maintain sterility. The C-arm may be moved in such a manner multiple times during a procedure, thereby moving the lower imaging head between the non-sterile 120 and sterile 118 zones multiple times and requiring repeated draping.

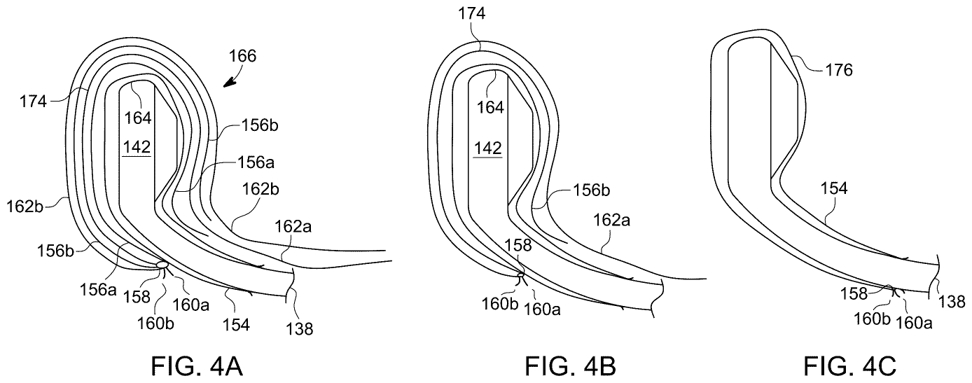

Embodiments of the present invention comprise a multi-layer drape that allows for multiple introductions and retractions of the lower imaging head into the sterile field while maintaining a sterile drape of the lower head. As illustrated in FIG. 4A, embodiments of the multi-layer drape 166 comprise a base drape layer 154 that surrounds the lower imaging head 142. The base layer may completely cover the imaging head and extend along a portion of the arcuate arm 138. The multi-layer drape may further comprise a first or inner removable drape layer 156a. The first or inner removable layer 156a may surround the imaging head 142 and attached to the base layer 154 at a bond site 158. A remnant or tail 160a of the first or inner removable layer may extend beyond the bond site 158.

A first tether 162a may be connected to the first or inner removable layer 156a at or adjacent the bond site 158. The first tether may comprise a strip of material and may wrap from the bond site over a top end 164 of the imaging head 142. The first tether may be attached to the first or inner removable layer at various points as the tether runs along the removable layer. The tether may be affixed to the removable layer by adhesive, ultrasonic bonding, or other attachments methods as would be understood by one of ordinary skill in the art. The tether 162 may comprise a separate piece of material attached to a surface or edge of the removable layer as illustrated, for example, in FIGS. 4A-4C. Alternatively, the tether may be integrally formed with the removable layer and in embodiments of the drape may comprise a tail extending from an edge of the removable layer. The tether may comprise the same material as the removable layer or may comprise a different material.

The multi-layer drape 166 may further comprise a second or outer removable layer 156b. The second or outer removable layer 156b may surround the imaging head 142 and attached to the first or inner removable layer 156a and base layer 154 at the bond site 158. A remnant or tail 160b of the second or outer removable layer may extend beyond the bond site 158. A second tether 162b may be connected to the second or outer removable layer 156b at or adjacent the bond site 158. The second tether may comprise a strip of material and may wrap from the bond site over a top end 164 of the imaging head 142. The second tether 162b may be attached to the second or outer removable layer 156b at various points as the tether runs along the removable layer. FIG. 4 shows the drape layers in cross-section, but it will be understood that each drape layer may surround the imaging head 142 and arcuate arm 138 on all sides except where the arcuate arm exits the drape.

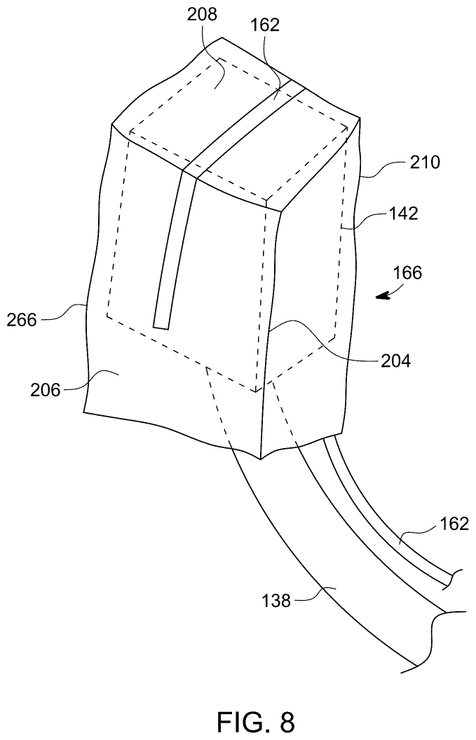

As illustrated in FIG. 8, for example, the multi-layer drape 166 may comprise a generally box-shaped drape 266 with one or more seams 204 that provide a shape that fits over the imaging head 142. The tether 162 may extend from a rear portion 206 of the box-shaped drape 266, over a top section 208, down a front section 210 of the drape, and from the drape to along the arcuate arm 138 or otherwise to the attachment point. Alternatively, the drape may comprise seams that form the drape into a generally conical, prismatic, or other shape that allows the drape to cover all or a portion of the imaging head 142.

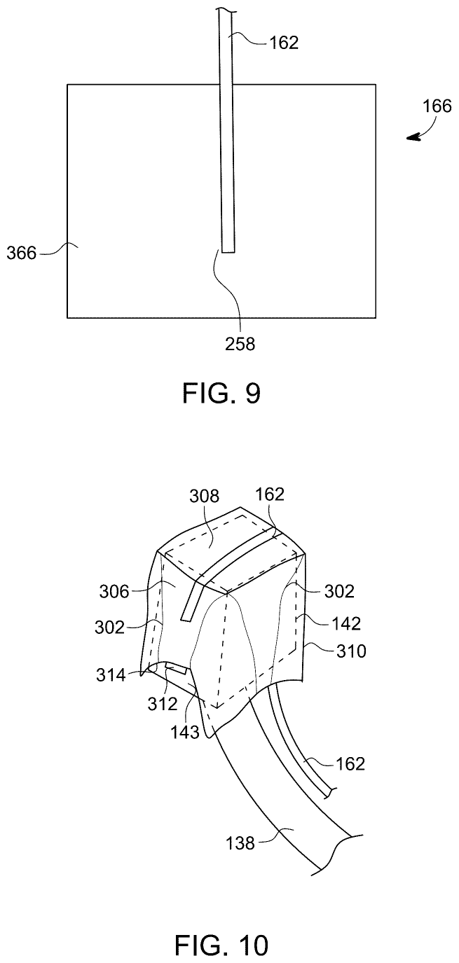

In further embodiments, as illustrated, for example, in FIGS. 9-10, the multi-layer drape 166 may comprise a flat, sheet-like drape 366, which may be draped over the imaging head 142. FIG. 9 illustrates the drape in a flat, pre-use configuration with tether 162 attached to a surface of each layer of the sheet at an attachment point 258. FIG. 10 illustrates the drape as positioned on the imaging head 142. One or more folds 302 in the sheet 366 may allow the drape to generally conform to the shape of the imaging head. The tether 162 may extend from a rear portion 306 of the folded drape 366, over a top section 308, down a front section 310 of the drape, and from the drape to along the arcuate arm 138 or otherwise to the attachment point. Tape 312 or other adhesive or attachment means may secure an edge 314 or surface of one or more layers of the drape a rear surface 143 of the imaging head 142.

As the imaging head is introduced into the sterile zone 118 for the first time, the second or outer removable layer 156b is removed, thereby exposing a sterile surface 174 of the first or inner removable layer 156a that was previously covered by the second or outer removable layer 156b. The second or outer removable layer is removed by pulling the second tether 162b, as explained in greater detail below. Perforations in the second or outer removable layer 156b allow the layer to detach from the first or inner removable layer 156a and base layer 154. The perforations may be positioned such that they allow detachment of the second or outer removable layer while preserving sterility of the first or inner removable layer when the imaging head 142 is reintroduced into the sterile zone 118. In embodiments of the drape, the perforations may be positioned at or adjacent to the attachment point 158, leaving the second or outer removable layer tail 160b attached to the remaining drape layers. Alternatively, perforations or other means of separation or detachment may be positioned at another portion of the drape. After lateral imaging is accomplished, the imaging head 142 may then be rotated below the sterile zone into the non-sterile zone.

It may then be advantageous to introduce the lower imaging head 142 into the sterile zone 118 a subsequent time. As the imaging head is introduced into the sterile zone 118 for a subsequent time, the first or inner removable layer 156a is removed, thereby exposing a sterile surface 176 of the base layer 154 that was previously covered by the first or inner removable layer 156a. The first or inner removable layer is removed by pulling the first tether 162a. Perforations in the first or inner removable layer 156a allow the layer to detach from the base layer 154. The perforations may be positioned such that they allow detachment of the removable layer while preserving sterility of the underlying layer when the imaging head is reintroduced into the sterile zone 118. In embodiments of the drape, the perforations may be positioned at or adjacent to the attachment point 158, leaving the first or inner removable layer tail 160a attached to the remaining drape layer. Alternatively, perforations or other means of separation or detachment may be positioned at another portion of the drape.

The embodiments discussed herein discuss a base layer 154 with first 156a and second 156b removable layers. However, other numbers of layers may be used. For example, the multi-layer drape may be provided with a single removable layer, from one to ten removable layers, or ten or more removable layers, each with an accompanying tether. In addition, embodiments of the drape may comprise a base layer 154 that is distinct from the one or more removable layers. Alternatively, the drape may comprise multiple layers without a distinct or separate base layer. In such embodiments, the lowest layer may be attached by tape, adhesive, or other means to the secure the drape to the imaging head 142.

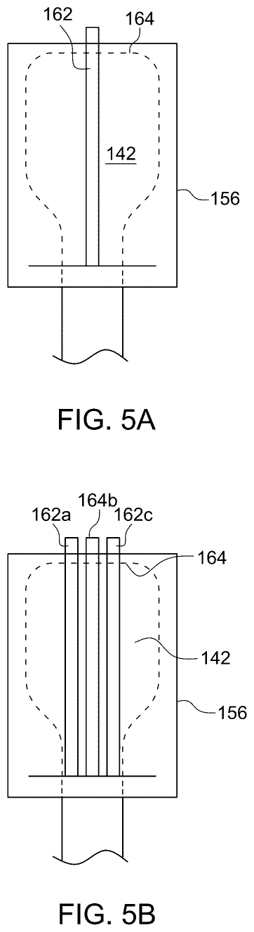

FIGS. 5A and 5B each shows a rear view of the lower imaging head 142 looking from the direction shown by arrow 178 in FIG. 3B. As illustrated in FIG. 5A, removable layers 156 may cover the imaging head 142, and tethers 162 may extend over a top end 164 of the imaging head. The tethers 162 for multiple layers 156 may be positioned in a stacked or overlapping configuration such that each tether extends over the top of an underlying tether. Alternatively, as illustrated in FIG. 5B, tethers 162a, 162b, 162c may be positioned in a spaced apart configuration such that the tethers do not overlap or only partially overlap. Depending on the number of layers, a combination of the configurations shown in FIGS. 5A and 5B may be used. For example, if nine removable layers are used, the tethers may be arranged in three stacks of three tethers.

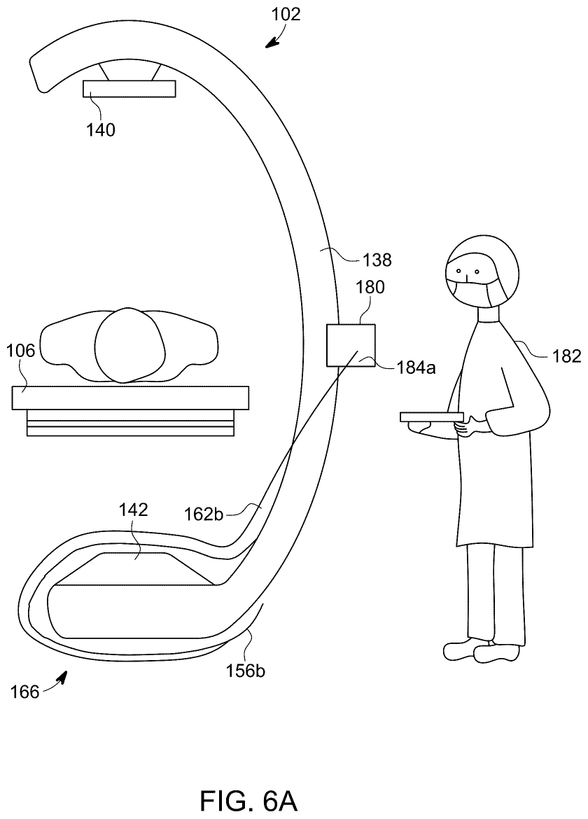

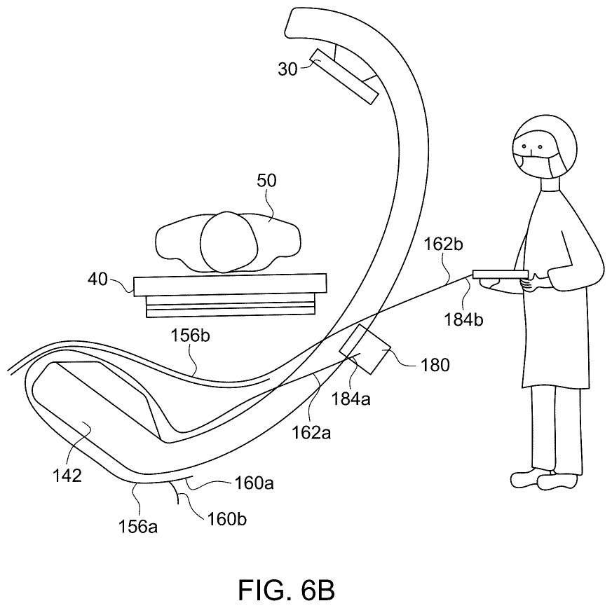

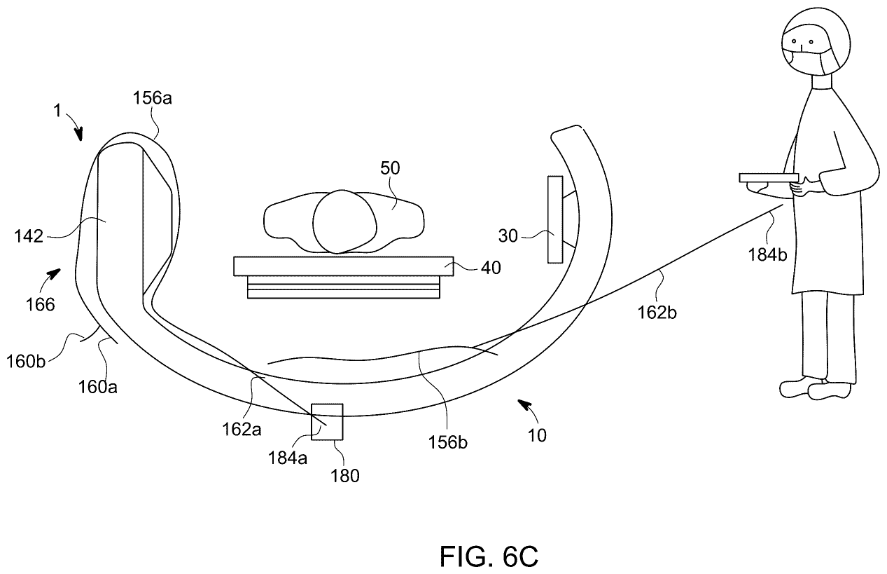

As illustrated in FIGS. 6A-C, in embodiments of the multi-layer drape 166, removal of a removable layer 156 may be accomplished by pulling the tether 162. In an initial state, as shown in FIG. 6A, the C-arm 102 is in an initial, vertical position with the lower image head 142 below the table 116. The second or outer removable layer 156b is wrapped around imaging head 142 with the tether 162b extending around the imaging head and to a common tether attachment or attachment point 180, which may be attached to or connected with a portion of the arcuate arm 138. In embodiments of the multi-layer drape, the attachment point is positioned on the C-arm 102 behind a rear portion 116 of the table 106 and outside of the sterile drape (110). Accordingly, a clinician 182 may be outside of the sterile zone 118 and thus not subject to the same sterility standards for personnel inside the zone.

Additional removable layers and tethers (see, e.g., FIGS. 6B, 156a, 162a) may be positioned in a similar manner. In a first removal step, the clinician 182 responsible for removing the second or outer removable layer 156b removes an end 184b of the tether 162b from the attachment point 180 and grasps the end 184b of the tether.

As illustrated in FIG. 6B, the C-arm 102 is then rotated. The C-arm may be rotated manually be the same clinician securing the tether or a different clinician or may be rotated by robotic, automatic, or other means. As the lower image head 142 rotates away from the clinician 182, the tether 162b pulls on the second or outer removable layer 156b and begins to detach it from the image head 142 and any lower layers still attached to the imaging head. The first or inner removable layer 156a and its accompanying tether 162a remain wrapped around the imaging head 142 with the end 184a of the tether 162 connected with the attachment point 180. As illustrated in FIG. 6C, the C-arm rotates to its final, lateral position. The second or outer removable layer 156b has been completely removed from the imaging head 142 except for the tail 160b, which in embodiments may remain attached to the multi-layer drape 166. The second or outer layer 156b and its accompanying tether 162b may then be collected and disposed of in an appropriate manner.

In embodiments of the drape, the second or outer layer of the drape may be removed in various ways. By way of example, the second or outer layer may be removed by a radiology technician, who may or may not have responsibility for rotation of the C-arm, by another clinician, or by other personnel such as a circulator, scrub nurse, scrub technician, or others. The clinician may be sterile or non-sterile. Alternatively, the second or outer layer may be removed without direct manipulation by a person. This may be accomplished by an automatic, computer controlled, or remote-controlled articulation mechanism or it may be accomplished automatically by movement of the C-arm from a first position to a second position.

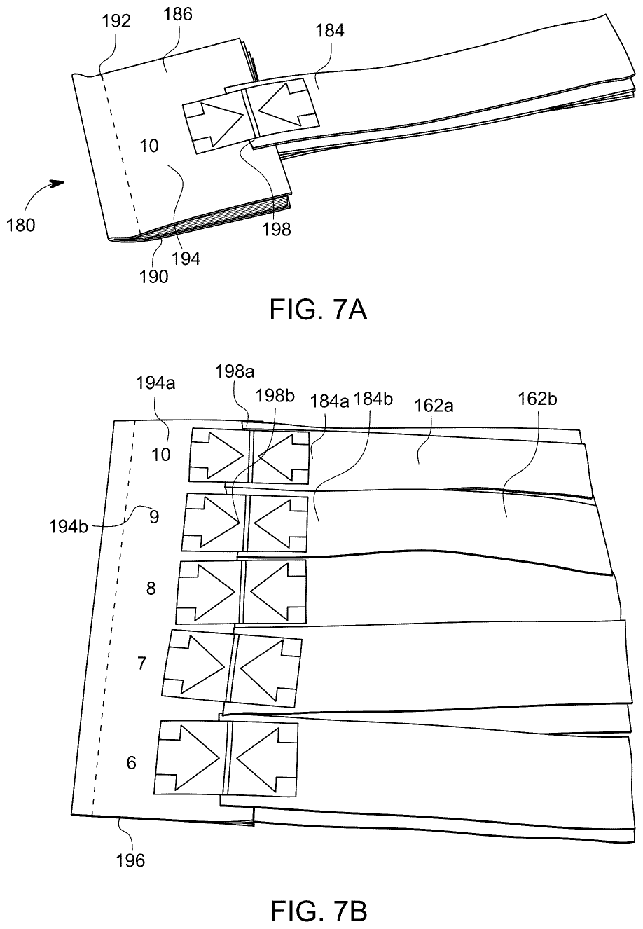

The attachment point 180 may provide for an organized arrangement of the tether ends 184. For example, as illustrated in FIG. 7A, the attachment point 180 may comprise a stack 190 of numbered labels 186, each attached to a tether end 184. Each numbered label 186 may be attached at one end 188 to the common stack 190 with perforations 192 that allow the outermost layer tether to be removed by tearing it from the stack. Alternatively, the tether end 184 may be removed from the label at a detachment point 198 with the label being separately removed from the stack. In embodiments of the drape, each label 186 may attach to and separate from the stack 190 using perforations, adhesive, bonding, tape, mechanical fasteners, or any other mechanism as would be apparent to one of ordinary skill in the art. The label 186 may comprise indicia 194. The indicia may comprise information regarding how many removable layers remain. Alternatively, as illustrated in FIG. 7B, the attachment point 180 may comprise a card 196 or other surface with multiple tether ends 184a, 184b attached in a spaced arrangement with detachment points 198a, 198b allowing removal of individual tethers 162a, 162b. The card 196 may comprise indicia 194a, 194b. The indicia may comprise information regarding how many removable layers remain. The attachment point may incorporate aspects of various embodiments. For example, the attachment point may comprise a first card having multiple tether attachments. The first card may be removably attached at a perforation 192 to a lower card also having multiple tether attachments.

As illustrated in FIGS. 6A-6C, the attachment point 180 may be attached to or connected with a portion of the arcuate arm 138. However, other attachment points may be used, including a separate stand or other equipment in the operating theater. The attachment point may be positioned behind the operating table 106 as illustrated in FIGS. 6A-6C. Alternatively, the attachment point may be positioned in another location, such as in front of the operating table, where it may be manipulated by a clinician positioned in front of the table. In further alternatives, the attachment point may be positioned at a head or foot of the table or at another position within the operating theater.

In embodiments of the multi-layer drape 166, each layer of the drape, including the base layer 154 and removable layers 156, may be made from a layer of a material that prevents the penetration of fluids. Embodiments of the drape layers may be made from a non-latex material, such as but not limited to, polyethylene, polypropylene, or combinations thereof. The drape layers may be comprised of films, of nonwovens, or of laminates of films and nonwovens. In one embodiment, the drape layers are biodegradable, and may comprise a processing aid to enhance biodegradability, one non-limiting example of which is Eco-One® manufactured by EcoLogic. Any suitable impermeable or impervious material could be used. In other embodiments the drape layers are made from two or more layers of material where each material layer is selected for its unique properties that are advantageous for a particular application. For example, one material layer may be made from a material that absorbs fluids while another layer is made from material that prevents penetration of fluids. In yet another embodiment, a single layer of absorbing material is used. Hence, embodiments are not limited to a specific material to form the drape layers. Embodiments the drape layers may be made of a transparent material. In other embodiments the drape layers may made of a select color, including translucent or tinted transparent material. In further embodiments, the color selected for the drape layers may be based on a color scheme that helps identify a desired use or application of the drape.

In embodiments of the multi-layer drape 166, each tether 162 attached to each removable layer 156 may comprise a material that is sufficiently strong to allow the removable layer to be detached without breaking or excessively stretching. As illustrated in FIGS. 4-5, the tether 162 may comprise a tape or tape-like configuration, being generally flat with width substantially greater than the thickness. Alternatively, the tether may be a cord, string, ribbon, tube, or other configuration. The tether material may comprise any appropriate material, including polymers such as polyethylene.

The written description set out above describes various features and aspects of various embodiments of drapes for use with C-arms or other equipment. However, it will be understood that the accompanying figures may illustrate additional features, elements, or functionality that, while not explicitly described, would be understood by a person having ordinary skill in the art. The figures and any features, elements, or functionality that would be understood therefrom form part of the disclosure of this application. While the present invention has been described with reference to one or more particular embodiments, those skilled in the art will recognize that many changes may be made thereto without departing from the spirit and scope of the present invention. Furthermore, components from one embodiment can be used in other non-exclusive embodiments. Each of these embodiments and obvious variations thereof is contemplated as falling within the spirit and scope of the invention.

What is claimed is:

1. A drape for use with a C-arm imaging unit, the drape comprising:
a base layer covering a surface of an imaging head of the imaging unit, wherein the imaging head is moveable between a retracted position outside a sterile zone and an extended position inside the sterile zone; and
a first removable layer at least partially covering the base layer, the first removable layer comprising a first tether extending from the first removable layer;
wherein the first removable layer is removed from the base layer by pulling on the first tether in conjunction with movement of the C-arm imaging head from the retracted position to the extended position.

2. The drape of claim 1 wherein removing the first removable layer from the base layer exposes a sterile surface of the base layer in conjunction with the imaging head moving into the sterile zone.

3. The drape of claim 1 wherein the base layer is secured to the C-arm.

4. The drape of claim 1 further comprising a tether attachment, wherein a first end of the first tether is connected with the first removable drape and a second end of the first tether is detachably connected to the tether attachment.

5. The drape of claim 4 wherein the tether attachment is connected to the C-arm at a position spaced apart from the imaging head.

6. The drape of claim 5 wherein the tether attachment is connected to the C-arm at a position spaced apart from the imaging head and outside the sterile zone.

7. The drape of claim 1 further comprising a second removable layer at least partially covering the first removable layer.

8. The drape of claim 7 wherein the second removable layer comprises a second tether.

9. The drape of claim 8 wherein the second removable layer is removed from the first layer by pulling on the second tether in conjunction with movement of the C-arm imaging head from the retracted position to the extended position in a first movement of the imaging head into the sterile zone.

10. The drape of claim 9 wherein the first removable layer is removed from the base layer by pulling on the first tether in conjunction with movement of the C-arm imaging head from the retracted position to the extended position in a second movement of the imaging head into the sterile zone.

11. The drape of claim 1 wherein the base layer comprises a substantially flat sheet of flexible material.

12. The drape of claim 1 wherein the first removable layer comprises a substantially flat sheet of flexible material.

13. The drape of claim 1 wherein the base layer comprises a flexible material having a first seam to at least partially conform the base layer to a shape of the imaging head.

14. The drape of claim 1 wherein the first removable layer comprises a flexible material having a first seam to at least partially conform the base layer to a shape of the imaging head.

15. The drape of claim 1 wherein the first removable layer is attached to the base layer.

16. The drape of claim 15 wherein the first removable layer is attached to the base layer by frangible connection.

17. The drape of claim 16 wherein the frangible connection comprises a perforation formed in the first removable layer.

18. The drape of claim 1 wherein the first tether is attached to the first removable layer.

19. The drape of claim 1 wherein the first tether is integrally formed with the first removable layer.

20. A drape for use with a C-arm imaging unit, the drape comprising:
a base layer covering a surface of an imaging head of the imaging unit, wherein the imaging head is moveable between a retracted position outside a sterile zone and an extended position inside the sterile zone;
a first removable layer at least partially covering the base layer, the first removable layer comprising a first tether extending from the first removable layer; and
at least one additional removable layer at least partially covering the first removable layer, the at least one additional removable layer comprising an attached tether;
wherein the at least one additional removable layer is removed from the base layer by pulling on the attached tether in conjunction with movement of the C-arm imaging head from the retracted position to the extended position in a first movement of the imaging head into the sterile zone; and
wherein the first removable layer is removed from the base layer by pulling on the first tether in conjunction with movement of the C-arm imaging head from the retracted position to the extended position in a second movement of the imaging head into the sterile zone.
